(19) United States
(12) Patent Application Publication
(10) Pub. No.: US 2025/0162909 A1
(43) Pub. Date: May 22, 2025

(54) A METHOD AND APPARATUS FOR CONTROLLING A COAGULANT DOSAGE IN A WATER TREATMENT SYSTEM

(71) Applicant: Kemira Oyj, Helsinki (FI)

(72) Inventors: Vesa-Matti TIKKALA, Helsinki (FI); Iiris JOENSUU, Helsinki (FI); Marjatta PIIRONEN, Helsinki (FI); Maria LUHTALA, Helsinki (FI)

(21) Appl. No.: 18/695,041

(22) PCT Filed: Sep. 26, 2022
(86) PCT No.: PCT/FI2022/050645

§ 371 Date: Mar. 25, 2024

(30) Foreign Application Priority Data
Sep. 30, 2021 (FI) 20216014

(51) Int. Cl. C02F 1/52

(52) U.S. Cl. CPC C02F 1/5209; C02F 1/5281; C02F 2209/003; C02F 2209/006; C02F 2209/11; C02F 2301/043

(57) ABSTRACT

A method of controlling a coagulant dosage in a water treatment system, such as for a tertiary wastewater treatment phase in a wastewater treatment system. The method comprises performing measurements indicative of post-precipitation downstream of a point at which an untreated first stream (101) and a second stream (102) treated with said coagulant (101) are joined, and using said measurements in adjusting the coagulant dosage.

### TECHNICAL FIELD

The present disclosure generally relates to water treatment. The disclosure relates particularly, though not exclusively, to a method and apparatus for optimizing a dosing of a chemical in a tertiary wastewater treatment process.

### BACKGROUND

This section illustrates useful background information without admission of any technique described herein representative of the state of the art.

Wastewater treatment plants are used to purify municipal sewage and/or industrial wastewater. In a conventional wastewater treatment plant, wastewater flows first to a mechanical preliminary treatment where different objects are removed from the wastewater, typically by one or more screens of different size. After the mechanical preliminary treatment, the wastewater flows into a primary treatment tank(s). The primary treatment is typically based on sedimentation of particles in the wastewater.

The wastewater then enters a secondary treatment that may be based on biological processes. They use bacteria which consume contaminants, in particular biodegradable organics, carbon and phosphorus and some nitrogen. The effluent from a secondary sedimentation tank may be clean enough to be released to a recipient, or it may undergo a further tertiary purification phase. The tertiary treatment typically comprises a solid-liquid separation step, which may be based on flotation. Certain chemicals, typically coagulant(s) and polymer(s) are dosed into a stream of wastewater prior to the wastewater enters solid-liquid separation tank(s), such as flotation tank(s). These chemicals precipitate the remaining dissolved organic compounds, which allows their removal in the solid-liquid separation step.

In the disclosed water treatment process, a correct dosing of chemicals into the water stream is important, for example concerning the environmental and cost-effective perspectives. To utilize a suitable amount of chemicals in the tertiary treatment is not straightforward and often the over- or underdosing is only noticed with a delay.

### SUMMARY

The appended claims define the scope of protection. Any examples and technical descriptions of apparatuses, products and/or methods in the description and/or drawings not covered by the claims are presented not as embodiments of the invention but as background art or examples useful for understanding the invention. It is an object of certain embodiments of the invention to provide an improved method for optimizing a chemical dosage, in particular a coagulant dosage, in a water treatment system or plant or at least to provide an alternative solution to existing technology.

According to a first example aspect of the invention there is provided a method of controlling at least a coagulant dosage in a water treatment system, comprising: performing measurements indicative of post-precipitation downstream of a joining point at which an untreated first stream, and a second stream treated with said coagulant are joined, the second stream being provided with a solid-liquid separation unit upstream of the joining point, and using said measurements in adjusting the coagulant dosage.

In certain embodiments, the water treatment system is selected from a group consisting of: a raw water treatment system, a wastewater treatment system, an internal industrial water treatment system and an internal industrial circulation water treatment system. Accordingly, in certain embodiments, the water treatment system is a wastewater treatment system. In certain other embodiments, the provided method is a method for controlling a coagulant dosage in a raw water treatment system. In certain embodiments, the provided method is a method for controlling a coagulant dosage in an internal industrial water circulation system. In this context, the term industrial may refer to pulp and paper industry, oil industry, mining industry, food industry, or to any other applicable industry.

In certain embodiments, the provided method is a method for a tertiary wastewater treatment phase in a wastewater treatment system. In certain embodiments, the tertiary treatment phase is a final cleaning process that improves wastewater quality prior to it is reuse, recycling or discharging to the environment.

In this context, the term wastewater may e.g. refer to municipal wastewater or to industrial wastewater. In this context, the term raw water may refer e.g. to surface water (such as water from a lake, sea or a river), to ground water, or to reused water.

Said performing measurements indicative of “post-precipitation downstream of a point” indicates the occurrence of post-precipitation downstream of the said point (measurements may be performed on both sides of the said point depending on the implementation).

In certain embodiments, the untreated first stream and the second stream originate from an outlet stream received from a preceding process phase (water purification phase), such as a wastewater secondary treatment phase that precedes the tertiary treatment phase. In certain embodiments, the secondary treatment phase is a phase based on biological processes.

The untreated first stream is defined as a stream that is not treated by said coagulant.

In this context, by post-precipitation is meant precipitation of material that occurs as a consequence of the untreated stream (first stream) meeting the stream (second stream) treated with the coagulant where said precipitation occurs due to a presence of chemical residue in the treated stream. In certain embodiments, said material refers to dissolved organic compounds. In certain embodiments, said chemical residue (or non-reacted chemical residue, wherein by “non-reacted” is meant non-reacted as far as the said precipitation of material is concerned) refers to remaining coagulant chemical(s) or coagulant-originated chemical(s), such as aluminum in the case of an aluminum containing coagulant or iron in the case of an iron containing coagulant. The said post-precipitation typically appears as turbidity in the joined stream.

In certain embodiments, the solid-liquid separation comprises a flotation process. In other embodiments, the solid-liquid separation is alternatively based on sedimentation. Accordingly, the said solid-liquid separation unit comprises a flotation unit or a sedimentation unit. The respective solid-liquid separation step is performed downstream of the said treatment with said coagulant (i.e., coagulant dosing step) and upstream of the said joining of the streams.

In certain embodiments, the method comprises:

decreasing the coagulant dosage in the event the post-precipitation occurs or the post-precipitation is increasing.

In certain embodiments, the measurements indicative of post-precipitation are turbidity measurements.

In certain embodiments, the measurements indicative of post-precipitation are turbidity measurements, the method comprising determining the post-precipitation based on measured difference in turbidity before and after the joining point at which the first and second stream are joined.

In certain embodiments, the turbidity before the joining point is determined as a flow-weighted average of the turbidity of the streams flowing to said joining point.

In certain embodiments, the method comprises:
performing a turbidity measurement downstream of the solid-liquid separation unit in the second stream to obtain the turbidity of the second stream;
performing a turbidity measurement downstream of the point at which the first stream and the second stream are joined to obtain the turbidity of the joined stream, and
adjusting the coagulant dosage based on a difference between the turbidity of the joined stream and the turbidity of the second stream or based on a change in the said difference.

In certain embodiments, the method comprises:
increasing the coagulant dosage in the event the turbidity of the second stream is greater than the turbidity of the joined stream.

In certain embodiments, the method comprises:
increasing the coagulant dosage in the event the turbidity of the second stream is greater than the turbidity of the joined stream and the difference in turbidity between the said streams increases.

In certain embodiments, the method comprises:
decreasing the coagulant dosage in the event the turbidity of the joined stream is greater than the turbidity of the second stream.

In certain embodiments, the method comprises:
decreasing the coagulant dosage in the event the turbidity of the joined stream is greater than the turbidity of the second stream and the difference in turbidity between the said streams increases.

In certain embodiments, the method comprises:
obtaining a difference between turbidity of the joined stream and turbidity of the second stream by retracting the turbidity of the second stream from the turbidity of the joined stream, and increasing the coagulant dosage (with which the second stream is treated) in the event the said difference is negative.

In certain embodiments, the method comprises:
obtaining a difference between turbidity of the joined stream and turbidity of the second stream by retracting the turbidity of the second stream from the turbidity of the joined stream, and increasing the coagulant dosage (with which the second stream is treated) in the event the said difference is negative and the said negative difference increases.

In certain embodiments, the method comprises:
obtaining a difference between turbidity of the joined stream and turbidity of the second stream by retracting the turbidity of the second stream from the turbidity of the joined stream, and decreasing the coagulant dosage (with which the second stream is treated) in the event the said difference is positive.

In certain embodiments, the method comprises:
obtaining a difference between turbidity of the joined stream and turbidity of the second stream by retracting the turbidity of the second stream from the turbidity of the joined stream, and decreasing the coagulant dosage (with which the second stream is treated) in the event the said difference is positive and the said positive difference increases.

In certain embodiments, the method comprises:
increasing the coagulant dosage in the event turbidity measurements in the second stream indicate an increase in turbidity.

In certain embodiments, the method comprises:
increasing the coagulant dosage in the event turbidity measurements in the second stream (downstream of the solid-liquid separation unit) indicate an increase in turbidity; and
decreasing the coagulant dosage in the event the post-precipitation occurs or increases.

In certain embodiments, the method comprises:
measuring a difference in turbidity between a point in the second stream before the joining point but after the solid-liquid separation unit and a point in the first stream before the joining point but after an optional solid-liquid separation unit; and using the said measured difference in determining the post-precipitation.

Accordingly, in certain embodiments, the method comprises:
measuring a difference in turbidity between outlets of the first and second streams; and
using the said measured difference in determining the post-precipitation.

In certain embodiments, the method comprises:
measuring a difference in turbidity between a point in the second stream before the joining point but after the solid-liquid separation unit and a point in the first stream before the joining point but after an optional solid-liquid separation step (i.e., the turbidity is measured from the outlets from the first and second streams), and in the event the turbidity at said point in the first stream is greater than the turbidity at said point in the second stream, defining post-precipitation as a difference between
   the turbidity of the joined stream, and
   a flow-weighted average turbidity of T₁ and T₂, where T₁ is the turbidity at said point in the first stream and T₂ is the turbidity at said point in the second stream.

In certain embodiments, the method comprises:
measuring a concentration of dissolved impurities in incoming water (such as wastewater incoming to the tertiary treatment phase);
switching on coagulant dosing when the concentration exceeds a predetermined limit; and
stopping dosing when the concentration returns below the limit.

In certain embodiments, the concentration of dissolved impurities is measured prior to the coagulant treatment.

In certain embodiments, said measuring a concentration of dissolved impurities comprises measuring a concentration of dissolved organic compounds and/or a concentration of dissolved inorganic material.

In certain embodiments, said measuring a concentration of dissolved impurities comprises measuring chemical oxygen demand, COD, concentration.

In certain embodiments, said measuring a concentration of dissolved impurities comprises measuring chemical oxygen demand, COD, concentration and/or measuring phosphorus concentration.

In certain embodiments, the method comprises:
applying a polymer dosage to the second stream when the coagulant dosing is on. In certain embodiments, the polymer dosage is a fixed dosage.

In certain embodiments, the method comprises:
removing suspended solids from the untreated first stream (prior to the joining of the streams) in a solid-liquid separation unit or step (such as in a flotation unit or step).

In certain embodiments, said coagulant is selected from a group consisting of: inorganic coagulants, preferably inorganic coagulants comprising iron containing salts, aluminum containing salts, magnesium containing salts, and any derivative thereof, preferably chlorides, sulphates, chlorosulphates, chlorohydrates, silicates, nitrates, and any derivate thereof, more preferably aluminum sulfate, polyaluminum sulfate, aluminum chloride, polyaluminum chloride, polyaluminum chlorosulfate, polyaluminum hydroxychlorosulfate, aluminum chlorohydrate, sodium aluminate, ferric sulfate, polyferric sulfate, ferric chloride, ferric chlorosulphate, polyferric chloride, ferrous sulfate, ferrous chlorosulphate, ferrous chloride, aluminum triformate, polyaluminum formate, polyaluminum nitrate, polyaluminum silicate, magnesium chloride, any derivative thereof, and any combination thereof, and (synthetic or natural) organic polymeric coagulants, the synthetic organic polymeric coagulant comprising preferably polyacrylamide, polyamine, polyDADMAC, polyethyleneimine, dicyandiamide and polyvinyl amine, the natural organic polymeric coagulant preferably comprising polysaccharide, such as starch, cellulose, guar gum, chitosan, dextran and the like, and polyphenolics, such as tannin and lignin, and any combinations of inorganic and organic polymeric coagulants. The organic polymeric coagulant may be anionic or cationic, preferably cationic. The polymeric coagulant comprises most preferably polyamine and/or polyDADMAC.

According to a second example aspect there is provided an apparatus, comprising: at least one processor; and at least one memory including computer program code, the at least one memory and the computer program code being configured, with the at least one processor, to cause the apparatus to perform the method of any of the first aspect or any related embodiment.

In certain embodiments, the apparatus comprises a control apparatus. In certain embodiments, the control apparatus comprises an analysis unit. In certain embodiments, the control apparatus further comprises measurement devices, for example measurement devices for turbidity measurements. In certain embodiments, the analysis unit comprises the said at least one processor.

According to a third example aspect of the present invention, there is provided a computer program comprising computer executable program code which when executed by a processor causes an apparatus to perform the method of the first aspect or any related embodiment.

According to a fourth example aspect there is provided a computer program product comprising a non-transitory computer readable medium having the computer program of the third example aspect stored thereon.

According to a fifth example aspect there is provided an apparatus comprising means for performing the method of the first aspect or any related embodiment.

Any foregoing memory medium may comprise a digital data storage such as a data disc or diskette, optical storage, magnetic storage, holographic storage, opto-magnetic storage, phase-change memory, resistive random access memory, magnetic random access memory, solid-electrolyte memory, ferroelectric random access memory, organic memory or polymer memory. The memory medium may be formed into a device without other substantial functions than storing memory or it may be formed as part of a device with other functions, including but not limited to a memory of a computer, a chip set, and a sub assembly of an electronic device.

Certain embodiments of the invention provide a method in which a chemical dosage optimization is provided with a COD, turbidity, and post-precipitation measurements from measurement points in at least the first stream, the second stream and an outlet stream. Said providing a chemical dosage optimization with a COD, turbidity and post-precipitation measurements comprises a control apparatus (or analysis unit) determining a chemical feed ON/OFF switch position and for calculating an optimal chemical dosage amount.

Different non-binding example aspects and embodiments have been illustrated in the foregoing. The embodiments in the foregoing are used merely to explain selected aspects or steps that may be utilized in different implementations. Some embodiments may be presented only with reference to certain example aspects. It should be appreciated that corresponding embodiments apply to other example aspects as well.

### BRIEF DESCRIPTION OF THE FIGURES

Some example embodiments will be described with reference to the accompanying figures, in which:

FIG. 1A schematically shows certain parts of a wastewater treatment plant according to certain example embodiments;

FIG. 1B schematically shows a certain modification to the wastewater treatment plant of FIG. 1A;

FIG. 1C schematically shows an additional turbidity measurement point in the wastewater treatment plant of FIG. 1A;

FIG. 2 schematically shows a block diagram of an apparatus according to an example embodiment;

FIG. 3 schematically shows certain parts of a wastewater treatment plant according to a modified example embodiment;

FIG. 4A schematically shows a flow chart according to certain example embodiments;

FIG. 4B schematically shows a flow chart according to further example embodiments;

FIG. 5A schematically shows dosage variation of the coagulant and the polymer feeds according to certain example embodiments; and

FIG. 5B schematically shows operation of chemical dosing analysis unit according to certain example embodiments.

### DETAILED DESCRIPTION

In the following description, like reference signs denote like elements or steps.

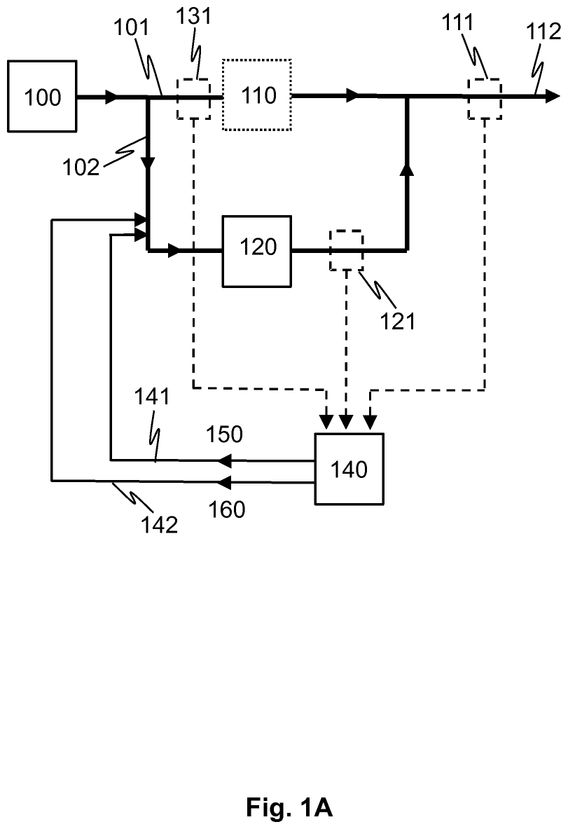

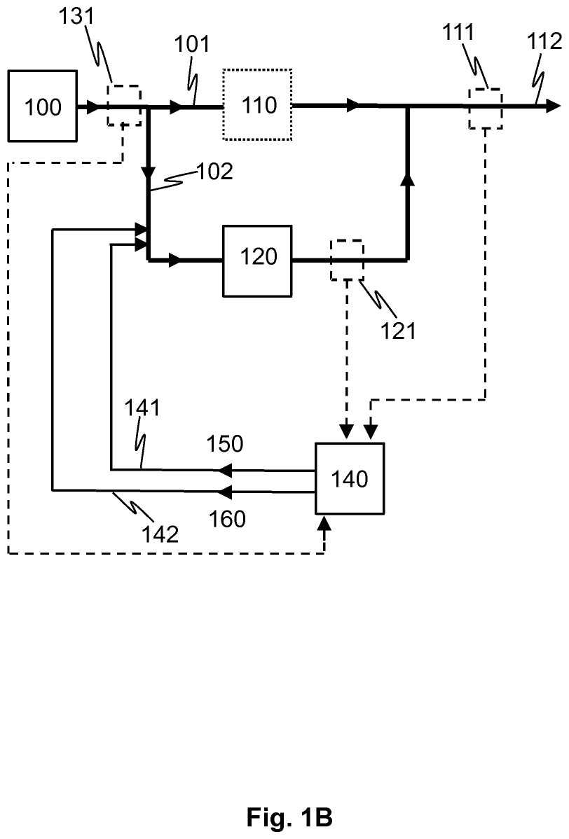

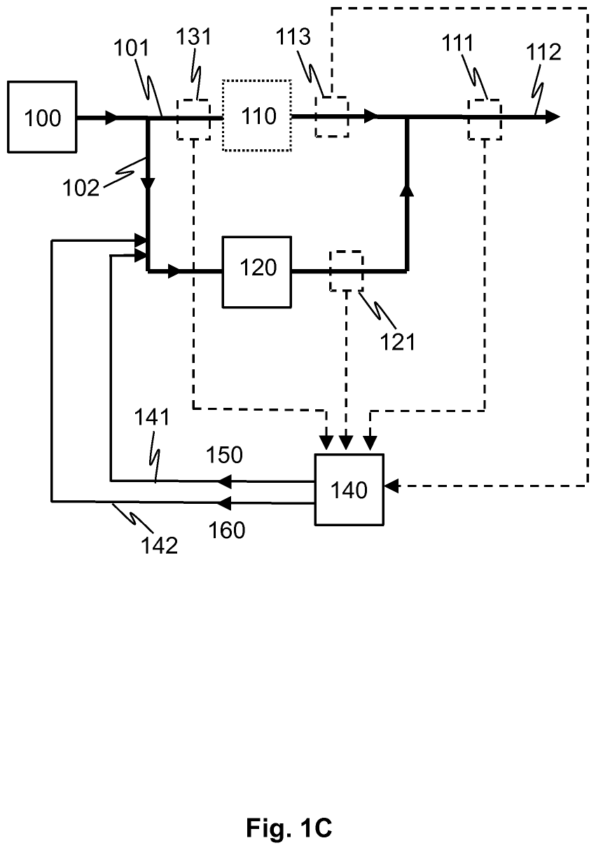

FIG. 1A schematically shows certain parts of a wastewater treatment plant or system according to certain example embodiments. Wastewater treatment generally comprises phases of primary, secondary and tertiary treatment. In certain embodiments, a major part of organic compounds is removed from the wastewater in a secondary treatment apparatus 100 by biological means, e.g. by using bacteria. Resulting sludge is removed in a secondary solid-liquid separation step typically based on sedimentation. An outlet from the secondary treatment apparatus 100 provides a water stream that flows into the tertiary wastewater treatment phase for further processing.

FIG. 1A shows that an inlet stream coming from the secondary treatment apparatus 100 to the tertiary treatment is divided to a first stream 101 and a second stream 102. In certain embodiments, the first stream 101 flows into a first solid-liquid separation unit or tank 110 for flotation. Further, the second stream 102 flows into a second solid-liquid separation unit or tank 120 for flotation.

It is generally known to improve the flotation result by dosing chemicals into a water stream. A coagulant dosage is generally used to precipitate dissolved inpurities, such as dissolved organic compounds and/or inorganic dissolved impurities like phosphorus from the treated water (raw water, wastewater, internal industrial water, and internal industrial circulation water). A polymer dosage increases the floc size of smaller flocs already formed by coagulant addition.

In certain embodiments, said coagulant and polymer are dosed into one of the streams while the other remains untreated by the said chemicals, and the dosage of the coagulant is optimized by using measurements indicative of post-precipitation downstream of a point at which the first and the second streams 101, 102 are rejoined. Accordingly, in certain embodiments, a coagulant 150 and a polymer 160 are dosed into the second stream 102 via a feed line 141 for the coagulant and a feed line 142 for the polymer. Precipitated material is removed in a solid-liquid separation step that occurs within the tank or unit 120. Instead of flotation, the solid-liquid tank 120 may be a sedimentation tank in other embodiments. The polymer dosage is optional, but has been shown useful in the increasing of the floc size.

The first stream 101 remains untreated by the said chemicals. Instead of removing dissolved organic compounds, the purpose of the solid-liquid separation step occurring in the tank or unit 110 (with the aid of flotation, or with the aid of sedimentation in other embodiments) therefore is to separate suspended solids from the first stream 101. Further, the solid-liquid separation tank or unit 110 has been drawn by a dotted line since in certain alternative embodiments the whole solid-liquid separation step is omitted in the first stream 101.

The first stream 101 and the second stream 102 combine (rejoin) after the solid-liquid separation step in units or tanks 110, 120 to form an outlet stream (or effluent) 112. Measurements indicative of post-precipitation are performed from the outlet stream 112 at a first measurement point 111. A control apparatus 140 receives measurements and controls chemical dosing based on the measurements. In the event the post-precipitation is increasing, the control apparatus reduces the coagulant dosing (since an increase in post-precipitation at the first measurement point 111 indicates overdosing). In an embodiment, the measurements indicative of post-precipitation in the outlet stream 112 are turbidity measurements.

In certain embodiments, turbidity is measured from the second stream 102 at a second measurement point 121 downstream of the second solid-liquid separation/flotation unit 120. The control apparatus 140 receives these measurements and increases the coagulant dosage in the event said measurements in the stream 102 indicate an increase in turbidity (since the increase in turbidity at the second measurement point 121 indicates underdosing). In certain embodiments, the coagulant dosage is increased in the event the measured turbidity exceeds a pre-defined limit. In certain embodiments, an incremental step upwards is dependent on a distance from the limit. Accordingly, in certain embodiments, the larger the measured turbidity the larger the incremental step.

In certain embodiments, the control apparatus 140 calculates differences between turbidity measurements at the first measurement point 111 and respective measurements at the second measurement point 121, and if the difference increases (which is an indication of overdosing), the control apparatus 140 decreases the coagulant dosage. In certain embodiments, the coagulant dosage is decreased in the event the turbidity difference exceeds a pre-defined limit. Also in these embodiments, an incremental step downwards may depend on the distance from the limit.

In certain embodiments, a COD concentration (chemical oxygen demand) (or other dissolved impurity concentration) is measured in a third measurement point 131 from an untreated stream. The third measurement point in certain embodiments is downstream of the point of division in the first stream 101 as shown in FIG. 1A. If the first stream comprises the solid-liquid separation unit 110, the measurement point 131 is preferably upstream of the said unit 110. In other embodiments, the third measurement point is upstream of the point of division in the inlet stream coming from the secondary treatment apparatus 100 as shown in FIG. 1B (FIG. 1B otherwise corresponds to FIG. 1A).

In certain embodiments, the control apparatus 140 receives the measurement(s) and determines whether the coagulant dosage is at all needed, i.e., whether it can be stopped. Accordingly, in certain embodiments, the control apparatus 140 switches on coagulant dosing when the incoming COD concentration (or other dissolved impurity concentration) exceeds a predetermined limit, and stops the dosing when the incoming COD concentration (or other dissolved impurity concentration) returns below the limit. The measurement of COD concentration (or other dissolved impurity concentration) therefore functions as an ON/OFF switch for the coagulant dosing. The ON/OFF switch concern the second stream 102 only (the first stream 101 remains untreated by the coagulant as mentioned in the foregoing).

In certain embodiments, as shown in FIG. 1C, the turbidity is measured from the outlets of both the first and second streams 101 and 102. Accordingly, in addition to the measurement point 121 in the second stream 102 also the outlet of the first stream 101 is provided with a measurement point (here: measurement point 113). First a difference in turbidity between the measurements points 113 and 121 is measured. If the difference T₁−T₂>0, or in certain embodiments, if the difference T₁−T₂ exceeds a pre-defined limit, then the post-precipitation is determined as a difference T₀-flow-weighted average of T₁ and T₂, where T₁ is the turbidity at point 113, and T₂ is the turbidity at point 121, and T₀ is the turbidity at point 111. With this kind of post-precipitation determination that takes account also the turbidity of the untreated stream 101 the reliability of the determination can be improved in certain embodiments. Otherwise, the adjustment of the coagulant dosing in these embodiments occurs similarly as described in the preceding (by replacing the turbidity 121 by the flow-weighted average turbidity).

Preferred methods of optimizing a dosage of a coagulant and an ON/OFF switch of the coagulant dosage have been disclosed in the preceding with reference to FIGS. 1A-1C. As to the polymer dosage, in an embodiment, the control apparatus 140 applies a fixed polymer dosage to the second stream 102 when the coagulant dosing is on, and the control apparatus 140 switched off also the polymer dosing when the coagulant closing is switched off. In other embodiments, the polymer dosage is not a fixed dosage but it may be for example dependent of the amount of the coagulant dosage.

The skilled person knows that depending on the coagulant used, the pH of the stream can be adjusted to optimize the performance. For that purpose, a chemical for controlling the pH can be dosed into the second stream 102 if appropriate (not shown). In certain embodiments, the used coagulant is an aluminum containing coagulant.

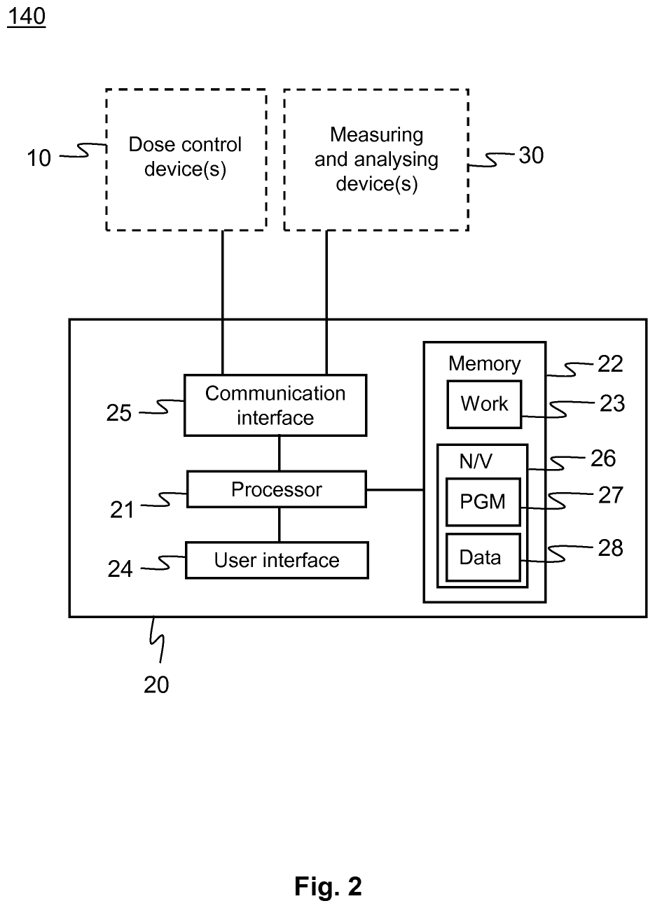

FIG. 2 shows a block diagram of the control apparatus 140 according to an embodiment. The apparatus 140 is suitable for implementing at least some of the operations disclosed in the preceding. The apparatus 140 comprises for example a general-purpose computer or server or some other electronic data processing apparatus 20. The apparatus 140 further comprises dose control device(s) 10, such as pump(s) and/or valve(s), and/or measuring and analyzing device(s) 30, such as turbidity measurement devices or a COD concentration analyzer. Alternatively, the devices 10 and/or 30 are implemented separately so that they do not form part of the apparatus 140.

The apparatus 20 comprises a communication interface 25, a processor 21, a user interface 24, and a memory 22.

The communication interface 25 comprises in an embodiment a wired and/or wireless communication circuitry, such as Ethernet, Wireless LAN, Bluetooth, GSM, CDMA, WCDMA, LTE, and/or 5G circuitry. The communication interface can be integrated in the apparatus 20 or provided as a part of an adapter, card or the like, that is attachable to the apparatus 20. The communication interface 25 may support one or more different communication technologies. The apparatus 20 may also or alternatively comprise more than one communication interface 25.

The processor 21 may be a central processing unit (CPU), a microprocessor, a digital signal processor (DSP), a graphics processing unit, an application specific integrated circuit (ASIC), a field programmable gate array, a microcontroller or a combination of such elements.

The user interface 24 may comprise a circuitry for receiving input from a user of the apparatus 20, e.g., via a keyboard, graphical user interface shown on the display of the apparatus 20, speech recognition circuitry, or an accessory device, such as a headset, and for providing output to the user via, e.g., a graphical user interface or a loudspeaker.

The memory 22 comprises a work memory 23 and a persistent (non-volatile, N/V) memory 26 configured to store computer program code 27 and data 28. The memory 26 may comprise any one or more of: a read-only memory (ROM), a programmable read-only memory (PROM), an erasable programmable read-only memory (EPROM), a random-access memory (RAM), a flash memory, a data disk, an optical storage, a magnetic storage, a smart card, a solid state drive (SSD), or the like.

The apparatus 20 may comprise a plurality of memories 26. The memory 26 may be constructed as a part of the apparatus 20 or as an attachment to be inserted into a slot, port, or the like of the apparatus 20 by a user or by another person or by a robot. The memory 26 may serve the sole purpose of storing data, or be constructed as a part of an apparatus 20 serving other purposes, such as processing data.

A skilled person appreciates that in addition to the elements shown in FIG. 2, the apparatus 20 may comprise other elements, such as microphones, displays, as well as additional circuitry such as an input/output (I/O) circuitry, memory chips, application-specific integrated circuits (ASIC), a processing circuitry for specific purposes such as a source coding/decoding circuitry, a channel coding/decoding circuitry, a ciphering/deciphering circuitry, and the like. Additionally, the apparatus 20 may comprise a disposable or rechargeable battery (not shown) for powering the apparatus 20 if an external power supply is not available. Further, it is noted that only one apparatus 20 is shown in FIG. 2, but certain embodiments may equally be implemented in a cluster of shown apparatuses.

The apparatus 20 performs the required algorithm(s) to control the ON/OFF switch operation and the operation for optimizing the coagulant dosage based on measurements or measurement data received from device(s) 30. In implementing these operations, the apparatus 20 controls the pumps and valve positions of the dose control device(s) 10 in an appropriate way.

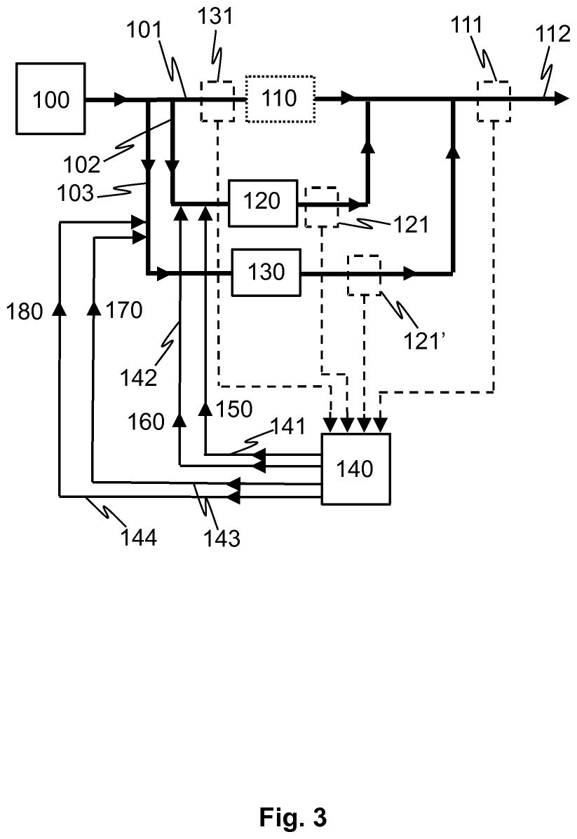

FIG. 3 schematically shows certain parts of a wastewater treatment plant according to a modified embodiment. The embodiment shown in FIG. 3 shows scalability of the preceding embodiments comprising a division of the inlet stream to three streams instead of division to two streams shown in the preceding (otherwise a reference is made to the preceding embodiments as to the structure and operation thereof). Accordingly, FIG. 3 presents an alternative, in which the first stream 101, the second stream 102 and a third steam 103 are divided from the inlet stream coming from the secondary treatment apparatus 100. A coagulant feed 170 and a polymer feed 180 to the third stream 103 occur prior to a solid-liquid separation/flotation unit 130 via a coagulant feed line 143 and a polymer feed line 144.

In certain embodiments, turbidity measurement data from the first measurement point 111, the second measurement point 121, and the third measurement point 131 are transferred to the control apparatus 140. Further, turbidity measurement data measured from the third stream 103 at a further measurement point 121′ downstream of the third solid-liquid separation/flotation unit 130 is transferred to the control device 140. In the control apparatus 140, an algorithm determines whether the coagulant feed 150 and polymer feed 160 is needed in the second stream 102 and whether the coagulant feed 170 and polymer feed 180 is needed in the third stream 103. Accordingly, an ON/OFF switch operation is implemented similarly as described in the preceding. In the control apparatus 140, the algorithm also determines the optimal dosage amount for the coagulant feeds 150 and 170 in a corresponding manner as disclosed in the preceding. Now only instead of the control apparatus 140 calculating differences between turbidity measurements at the first measurement point 111 and respective measurements at the second measurement point 121, the differences in turbidity are calculated also between measurements at the first measurement point 111 and respective measurements at the measurement point 121′, etc.

As shown in FIG. 1B in the preceding, the third measurement point for measuring COD concentration may be also here, alternatively, upstream of the point of division of the inlet stream.

In yet alternative embodiments, the number of (divided) adjacent streams may be more than three, such as four or more.

In alternative embodiments, as indicated in the preceding, each stream divided from the inlet stream need not contain a solid-liquid separation/flotation unit. For example, it is possible to implement a tertiary treatment apparatus comprising only one solid-liquid separation/flotation unit having an outlet and the apparatus further comprising a side stream (without a solid-liquid separation/flotation unit) joining the solid-liquid separation/flotation unit outlet to form a joined outlet stream, and to perform measurements indicative of post-precipitation from the joined outlet stream and from the outlet of the solid-liquid separation/flotation unit (and from the outlet of the side stream as the case may be).

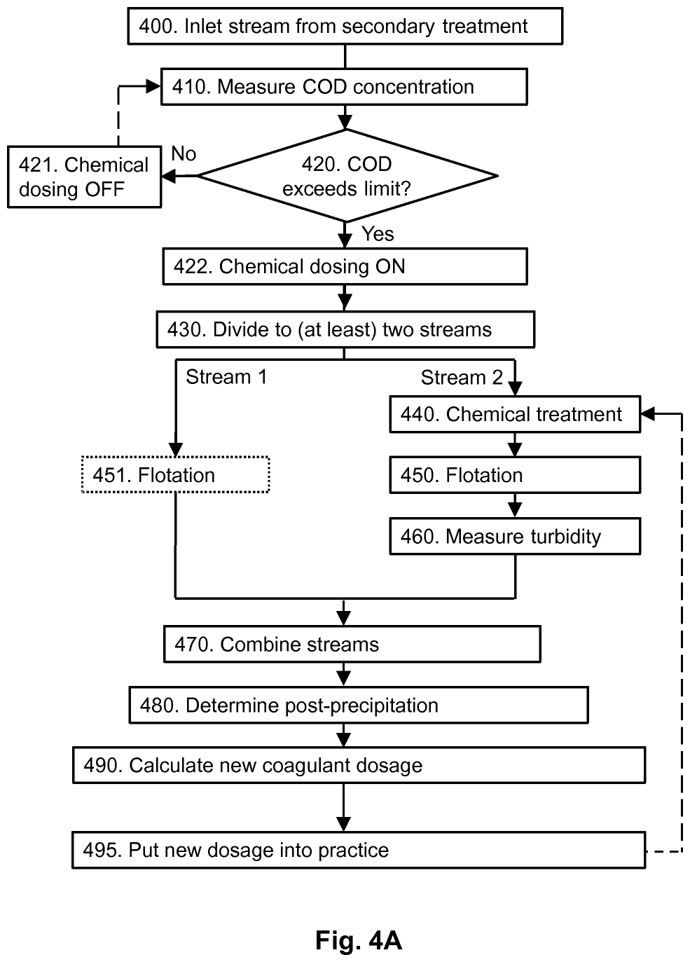

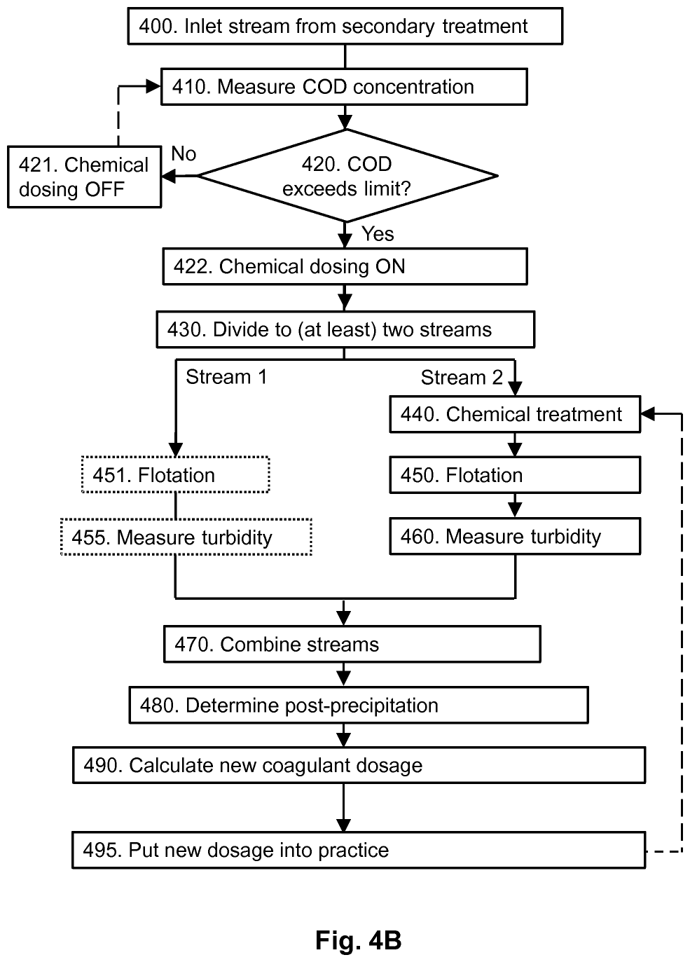

FIG. 4A schematically shows flow chart according to certain process embodiments. In step 400, the inlet stream is received from the secondary treatment phase to the tertiary treatment apparatus (or system). In step 410, the COD concentration is measured from an untreated stream. Whether or not the COD concentration exceeds a predefined limit is determined in step 420. If not, the chemical dosing is kept off (or switched OFF) in step 421. The step 410 is then entered anew after a predefined period of time has passed. If the COD concentration exceeds a predefined limit, the chemical dosing is kept on (or switched ON). The inlet stream is divided into (at least) two streams (phase 430). Chemical (coagulant(s) and polymer(s)) is dosed into one stream (Stream 2) in step 440 prior to the flotation/solid liquid separation step 450 while the other stream (Stream 1) only undergoes flotation/solid liquid separation (step 451), or in certain embodiments the step 451 may be omitted. In step 460, turbidity is measured from the flotation outlet of Stream 2. The streams are combined in step 470. The post-precipitation is determined in step 480 e.g. based on turbidity measurements as shown in the preceding. A new coagulant dosage is determined based on the turbidity measurements and the post-precipitation determination in step 490. For example, if the turbidity from the flotation/solid liquid separation outlet of Stream 2 has been increased, the coagulant dosage is stepwise increased, whereas if the post-precipitation has been increased and the turbidity at the flotation/solid liquid separation outlet has not been increased, the coagulant dosage is stepwise decreased. In step 495, the new coagulant dosage is put into practice with a link to step 440 from which the method continues towards new measurements.

FIG. 4B shows an alternative to FIG. 4A containing the further turbidity measurement step 455 (at point 113) as disclosed in the preceding in the context of FIG. 1C. The post-precipitation in the step 480 is determined by using the flow-weighted average as explained in the preceding in the context of FIG. 1C. Otherwise, the embodiment shown in FIG. 4B operates similarly as the embodiment shown in FIG. 4A.

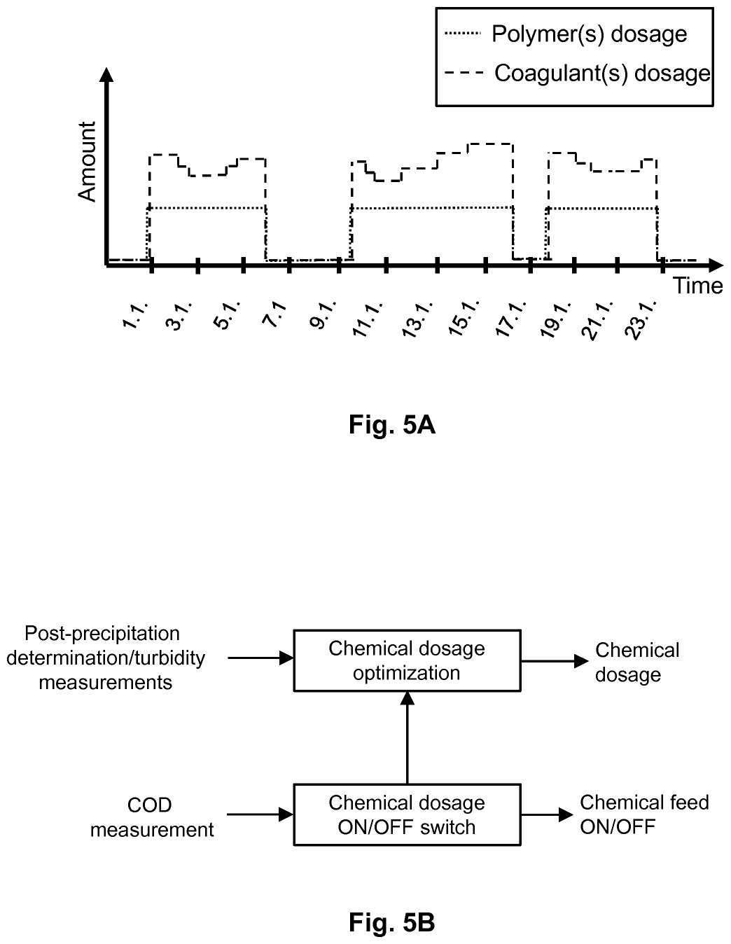

FIG. 5A schematically shows dosage variation of the coagulant(s) and the polymer(s) feeds. In certain embodiments, the polymer(s) dosage amount is constant, and the coagulant(s) dosage amount is optimized based on the conditions in the process. In FIG. 5A, the OFF switch is activated based on the COD amount measured from the first stream and the dosage curves for both chemicals drop to the zero-level. As the COD level increases in the process, the said measurement in the first stream detects it, and allows chemicals to be fed into the process. Accordingly, an ON switch is activated. The amount of coagulant(s) varies, which is seen in FIG. 5A. The variation of the coagulant(s) dosage amount is due to the optimization that occurs in the control apparatus. The turbidity measurements and the post-precipitation determination are used to generate information for the appropriate algorithm to adjust the coagulant(s) dosage amount accordingly.

The method provides a fast or near real-time response to adjust chemical dosing according to the process needs.

FIG. 5B schematically shows the chemical (or coagulant) dosing adjustment process according to certain example embodiments. The FIG. 5B provides additional clarification to the analysis unit operation already discussed in the preceding. In certain embodiments, the COD measurement provides the information from the process that is used to determine the ON/OFF switch for the chemical dosing. The turbidity measurements and the post-precipitation determination provide information from the process, and these are used to adjust (or optimize) the chemical dosage amount.

Without limiting the scope and interpretation of the patent claims, certain technical effects of one or more of the example embodiments disclosed herein are listed in the following. A technical effect is providing a near real-time method for optimizing a dosing of a chemical/coagulant in a water treatment process, such as a tertiary wastewater treatment process. Another technical effect is the ability to minimize a chemical content in an effluent discharged from the water treatment system to nature.

Various embodiments have been presented. It should be appreciated that in this document, words comprise, include and contain are each used as open-ended expressions with no intended exclusivity.

The foregoing description has provided by way of non-limiting examples of particular implementations and embodiments a full and informative description of the best mode presently contemplated by the inventors for carrying out the invention. It is however clear to a person skilled in the art that the invention is not restricted to details of the embodiments presented in the foregoing, but that it can be implemented in other embodiments using equivalent means or in different combinations of embodiments without deviating from the characteristics of the invention. For example, the disclosed embodiments can be used to describe a method and apparatus of controlling a coagulant dosage in a raw water treatment system or in an industrial water circulation system. In those embodiments, the secondary treatment apparatus 100 may simply be renamed as an apparatus performing a “preceding” process phase.

Furthermore, some of the features of the afore-disclosed example embodiments may be used to advantage without the corresponding use of other features. As such, the foregoing description shall be considered as merely illustrative of the principles of the present invention, and not in limitation thereof. Hence, the scope of the invention is only restricted by the appended patent claims.

1. A method of controlling a coagulant dosage in a water treatment system, comprising:
performing measurements indicative of post-precipitation downstream of a joining point at which an untreated first stream, and a second stream treated with said coagulant are joined, the second stream being provided with a solid-liquid separation unit upstream of the joining point, and
using said measurements in adjusting the coagulant dosage.

2. The method of claim 1, comprising:
decreasing the coagulant dosage in the event the post-precipitation occurs or the post-precipitation is increasing.

3. The method of claim 1, wherein the measurements indicative of post-precipitation are turbidity measurements, the method comprising determining the post-precipitation based on measured difference in turbidity before and after the joining point at which the first and second stream are joined.

4. The method of claim 1, comprising:
performing a turbidity measurement downstream of the solid-liquid separation unit in the second stream to obtain the turbidity of the second stream;
performing a turbidity measurement downstream of the point at which the first stream and the second stream are joined to obtain the turbidity of the joined stream, and
adjusting the coagulant dosage based on a difference between the turbidity of the joined stream and the turbidity of the second stream or based on a change in the said difference.

5. The method of claim 1, comprising:
increasing the coagulant dosage in the event the turbidity of the second stream is greater than the turbidity of the joined stream.

6. The method of claim 1, comprising:
increasing the coagulant dosage in the event the turbidity of the second stream is greater than the turbidity of the joined stream and the difference in turbidity between the said streams increases.

7. The method of claim 1, comprising:
decreasing the coagulant dosage in the event the turbidity of the joined stream is greater than the turbidity of the second stream.

8. The method of claim 1, comprising:
decreasing the coagulant dosage in the event the turbidity of the joined stream is greater than the turbidity of the second stream and the difference in turbidity between the said streams increases.

9. The method of claim 1, comprising:
increasing the coagulant dosage in the event turbidity measurements in the second stream indicate an increase in turbidity.

10. The method of claim 1, comprising:
increasing the coagulant dosage in the event turbidity measurements in the second stream indicate an increase in turbidity; and
decreasing the coagulant dosage in the event the post-precipitation occurs or increases.

11. The method of claim 1, comprising:
measuring a concentration of dissolved impurities in incoming water;
switching on coagulant dosing when the concentration exceeds a predetermined limit; and
stopping dosing when the concentration returns below the limit.

12. The method of claim 11, wherein said measuring a concentration of dissolved impurities comprises measuring chemical oxygen demand, COD, concentration and/or measuring phosphorus concentration.

13. The method of claim 1, comprising:
applying a polymer dosage to the second stream when the coagulant dosing is on.

14. The method of claim 1, comprising:
removing suspended solids from the untreated first stream in a solid-liquid separation unit or step.

15. The method of claim 1, comprising:
measuring a difference in turbidity between outlets of the first and second streams; and
using the said measured difference in determining the post-precipitation.

16. The method of claim 1, wherein the water treatment system is selected from a group consisting of: a raw water treatment system, a wastewater treatment system, an internal industrial water treatment system, and an internal industrial circulation water treatment system.

17. The method of claim 1, wherein the provided method is a method for a tertiary wastewater treatment phase in a wastewater treatment system.

18. The method of claim 1, wherein said coagulant is selected from a group consisting of: inorganic coagulants, preferably inorganic coagulants comprising iron containing salts, aluminum containing salts, magnesium containing salts, and any derivative thereof, preferably chlorides, sulphates, chlorosulphates, chlorohydrates, silicates, nitrates, and any derivate thereof, more preferably aluminum sulfate, polyaluminum sulfate, aluminum chloride, polyaluminum chloride, polyaluminum chlorosulfate, polyaluminum hydroxychlorosulfate, aluminum chlorohydrate, sodium aluminate, ferric sulfate, polyferric sulfate, ferric chloride, ferric chlorosulphate, polyferric chloride, ferrous sulfate, ferrous chlorosulphate, ferrous chloride, aluminum triformate, polyaluminum formate, polyaluminum nitrate, polyaluminum silicate, magnesium chloride, any derivative thereof, and any combination thereof, and synthetic and natural organic polymeric coagulants, the synthetic organic polymeric coagulant comprising preferably polyacrylamide, polyamine, polyDADMAC, polyethyleneimine, dicyandiamide and polyvinyl amine, the natural organic polymeric coagulant preferably comprising polysaccharide, such as starch, cellulose, guar gum, chitosan, dextran and the like, and polyphenolics, such as tannin and lignin, and any combinations of inorganic and organic polymeric coagulants.

19. An apparatus, comprising:
at least one processor; and
at least one memory including computer program code, the at least one memory and the computer program code being configured, with the at least one processor, to cause the apparatus to perform the method of claim 1.

20. A computer program comprising computer executable program code which when executed by at least one processor causes an apparatus to perform the method of claim 1.
